(19) United States
(12) Patent Application Publication
(10) Pub. No.: US 2023/0054162 A1
(43) Pub. Date: Feb. 23, 2023

(54) METHODS FOR PRODUCING OIL-IN-WATER EMULSIONS

(71) Applicant: Fresenius Kabi Austria GmbH, Graz (AT)

(72) Inventors: Andreas Heinrich JUNG, Graz (AT); Christoph KOTH, Graz (AT); Peter Gerhard STADLER, Graz (AT)

(21) Appl. No.: 17/787,478

(22) PCT Filed: Dec. 18, 2020
(86) PCT No.: PCT/EP2020/086956

§ 371 Date: Jun. 20, 2022

(30) Foreign Application Priority Data
Dec. 20, 2019 (EP) 19218741.7

(51) Int. Cl. A61K 9/107; A61K 9/00; A61K 47/44

(52) U.S. Cl. CPC A61K 9/1075; A61K 9/0029; A61K 47/44

(57) ABSTRACT

The present invention relates to a highly efficient method for producing oil-in-water emulsions, preferably for parenteral administration, which method is characterized in that large parts of the water phase are added only after the energy-intensive homogenization of the pre-emulsion, i.e. an emulsion having low water content is first produced and said emulsion is subsequently “diluted”.

### FIELD OF THE INVENTION

The present invention relates to the production of oil-in-water emulsions, the emulsions produced in accordance with the method according to the invention as well as their use as a medication or in the provision of parenteral nutrition.

### BACKGROUND OF THE INVENTION

Emulsions are disperse systems of two immiscible liquids. A distinction is made here between the inner or dispersed phase, which is divided into discrete droplets, and the outer phase, the dispersant.

Such systems are not stable without other additives, but can, for example, be stabilized by adding emulsifiers. Emulsifiers belong to the surface-active substances. They are attached to the phase boundary, facilitate the formation of the droplets, which forms the inner phase in the outer phase, and counteract the phase separation.

Oil droplets are present dispersed in a water phase in oil-in-water emulsions.

Oil-in-water emulsions are produced for the most varied of purposes, e.g. as emulsions for use in parenteral nutrition or as a basis for emulsions containing drugs, such as for example propofol.

Oil-in-water emulsions for parenteral administration must, at least when they are administered in larger volumes, also have an osmolality as similar as possible to the blood, i.e. an isotonic agent must be added to them.

A disadvantage of these oil-in-water emulsions is that their production, in particular in the quality required for parenteral administration, is complex and subject to a very high rejection rate.

Pharmaceutical oil-in-water emulsions for parenteral application are produced in a two-stage process as standard, in which first from the water phase (containing the isotonic agent), an emulsifier and the oil phase a pre-emulsion is produced, in which the diameter of the oil droplets is in the micrometer range. The pre-emulsion can, for example, be produced by means of a rotor-stator disperser.

An emulsion is then obtained from the pre-emulsion, for example by a multi-stage high-pressure homogenization process. Oil droplets are minimized in this process such that this emulsion then also meets the requirements for parenterally administered preparations, among others, with regards to the droplet size distribution.

In the case of oil-in-water emulsions for parenteral administration, the average diameter of the oil droplets may not exceed 0.5 μm for physiological reasons (owing to the anatomy and the size of the blood vessels).

Additionally, in the case of parenterally administered emulsions, the PFAT₅ value (the percentage proportion of oil droplets within the oil phase of an oil-in-water emulsion with a diameter of more than 5 μm) must be below 0.05% (see USP 729).

However, it very often happens that this limit value is exceeded. In practice, a PFAT₅ value of above 0.05% is found in roughly 20 to 30% of the manufactured emulsions. These batches have to be destroyed which causes huge economic losses.

Additionally, the methods currently practiced as standard are very time-consuming and energy-intensive.

The object of the present invention is therefore to provide a time and energy-efficient method for producing oil-in-water emulsions, which allows emulsions to be reliably and reproducibly obtained, which meet the high requirements for parenterally administered compositions.

### SUMMARY OF THE INVENTION

This object is surprisingly achieved in that, in the production methods for oil-in-water emulsions according to the invention, large parts of the water phase are added only after the energy-intensive homogenization of the pre-emulsion, i.e. an emulsion with lower water content is first produced, which is then diluted.

The invention thus relates to a method for producing an oil-in-water emulsion, comprising a water phase and 1 to 40, preferably 5 to 30, most preferably 10 to 30% of an oil phase, in relation to the total weight of the emulsion, with the method comprising the following steps:
a) providing an oil phase, comprising one or a plurality of oils and optionally at least one pharmaceutically acceptable antioxidant and/or at least one pharmaceutically acceptable co-emulsifier,
b) providing a water phase 1, comprising water and optionally at least one pharmaceutically acceptable co-emulsifier and/or at least one substance for setting the pH value and/or at least one pharmaceutically acceptable preservative and/or at least one pharmaceutically acceptable isotonic agent, with the isotonic agent being present in a concentration of at most 18%, preferably 15%, particularly preferably at most 14.3%, in relation to the total weight of the water phase 1,
c) producing a pre-emulsion by mixing the oil phase, provided in step a), with the water phase 1, provided in step b),
d) producing a first emulsion by homogenizing the pre-emulsion, provided in step c),
e) providing a water phase 2, comprising water and optionally at least one pharmaceutically acceptable isotonic agent and/or at least one substance for setting the pH value and/or at least one pharmaceutically acceptable preservative,
f) producing the emulsion by mixing the first emulsion, provided in step d), with the water phase 2, provided in step e) and
g) sterilizing the emulsion, obtained in step f),
   with the emulsion being filled into a suitable container before or after being sterilized,
   with at least one pharmaceutically acceptable emulsifier being added in step a) and/or in step b), and
   with the water phase 1, provided in step b), providing no more than 70%, preferably no more than 50%, particularly preferably no more than 30% and most preferably no more than 20% of the total amount of water contained in the emulsion.

The invention also relates to the oil-in-water emulsion obtained according to this method and its use as a medication or in the provision of parenteral nutrition.

Additionally, the invention relates to the first emulsion obtained in step d).

### DETAILED DESCRIPTION OF THE INVENTION

In the case of the known methods for producing oil-in-water emulsions, a pre-emulsion is first produced by intensive mixing from the entire water phase, to which were previously added, if applicable, at least one isotonic agent as well as, if applicable, at least one preservative, if applicable, at least one agent for setting the pH value and/or other water-soluble substances, and from the entire oil phase, to which were previously added, if applicable, at least one antioxidant and, if applicable, other lipophilic components, in the presence of at least one emulsifier, which was previously added depending on its chemical nature either to the oil or water phase.

By homogenizing, for example by means of high-pressure homogenizers, counter-jet dispersers or using ultrasound, this pre-emulsion is transferred into an emulsion in which the average size of the oil droplets is significantly reduced compared with the pre-emulsion.

These processes are time-consuming and energy-intensive and, under certain circumstances, require a large-volume device.

It has now surprisingly been found that, when producing the pre-emulsion and obtaining the emulsion, parts of the water phase can be omitted and added only after the emulsifying, without negatively affecting the quality of the emulsions, provided the concentration of the isotonic agent in the water phase 1 during the emulsifying process does not exceed 18%, preferably 15%, particularly preferably 14.3%, in relation to the total weight of the water phase 1.

The method according to the invention for producing oil-in-water emulsions, comprising a water phase and 1 to 40%, preferably 5 to 30%, most preferably 10 to 30% of an oil phase, in relation to the total weight of the emulsion, comprises the following steps:

a) providing an oil phase, comprising one or a plurality of oils and optionally at least one pharmaceutically acceptable antioxidant and/or at least one pharmaceutically acceptable co-emulsifier,

b) providing a water phase 1, comprising water, optionally at least one pharmaceutically acceptable co-emulsifier and/or at least one substance for setting the pH value and/or at least one pharmaceutically acceptable preservative and/or at least one pharmaceutically acceptable isotonic agent, with the isotonic agent being present in a concentration of at most 18%, preferably 15%, particularly preferably at most 14.3%, in relation to the total weight of the water phase 1,

c) producing a pre-emulsion by mixing the oil phase, provided in step a), with the water phase 1, provided in step b),

d) producing a first emulsion by homogenizing the pre-emulsion provided in step c),

e) providing a water phase 2, comprising water, optionally at least one pharmaceutically acceptable isotonic agent and/or at least one substance for setting the pH value and/or at least one pharmaceutically acceptable preservative,

f) producing the emulsion by mixing the first emulsion, provided in step d), with the water phase 2, provided in step e) and

g) sterilizing the emulsion, obtained in step f),

with the emulsion being filled into a suitable container before or after being sterilized,

with at least one pharmaceutically acceptable emulsifier being added in step a) and/or in step b), and

with the water phase 1, provided in step b), providing no more than 70%, preferably no more than 50%, particularly preferably no more than 30% and most preferably no more than 20% of the total amount of water contained in the emulsion.

The present invention also relates to the oil-in-water emulsions produced according to this method and their use as a medication or in the provision of parenteral nutrition.

The present invention also relates to the emulsions obtained in step d) of the method which can be packaged, stored and transported prior to their further processing in the steps e), f) and g) according to the invention.

The present invention also relates to the emulsions obtained in step d) for use in the further processing according to the steps e), f) and g) of the method according to the invention.

The method according to the invention is advantageous in multiple respects:

Firstly, capacity in the homogenization tool is saved due to the volume of the water phase being reduced, which results in shortened homogenization times and therefore a notably improved yield/efficiency. (Thus, for example, a high-pressure homogenizer requires roughly one hour to homogenize 1000 kg of a pre-emulsion.)

Secondly, since smaller masses have to be processed in the energy-intensive work steps, time and energy are saved. In addition to the above-mentioned increase in efficiency during homogenization, the heating of the water phase in particular is more efficient in terms of energy and time due to its reduced mass.

Thirdly, there is also a significant savings potential in the case of the vessel and room sizes within the production facilities.

The method surprisingly also delivers better quality emulsions. Improvements can in particular be found with regard to the droplet size distribution.

Thus, the droplet size distribution is more reproducible and narrower in the emulsions, which are produced in accordance with the method according to the invention.

Additionally, the PFAT₅ values are notably lower in the emulsions produced in accordance with the method according to the invention.

The emulsions produced in accordance with the method according to the invention preferably have a PFAT₅ value of below 0.05%, particularly preferably of below 0.04%, more preferably of below 0.03% and most preferably of below 0.02%.

The emulsions produced in accordance with the method according to the invention preferably have an average PFAT₅ value of below 0.035%, particularly preferably of below 0.030% and particularly preferably of below 0.025%. Quite particularly preferably, the emulsions produced in accordance with the method according to the invention have, more preferably of below 0.020%, even more preferably of below 0.015% and most preferably of below 0.010%. A sample size of at least 10 must be used to determine this average value.

In the case of production according to the known standard methods, PFAT₅ values of above 0.05% are often found in practice. Surprisingly, the method according to the invention reduces the PFAT₅ value far below 0.05% and the average PFAT₅ value far below 0.035%. The present invention also relates to a system for producing an oil-in-water emulsion, with the oil-in-water emulsion comprising a water phase and 1 to 40%t, preferably 5 to 30%, most preferably 10 to 30% of an oil phase, in relation to the total weight of the emulsion, comprising

a) a first vessel for providing an oil phase comprising the following components: one or a plurality of oils and optionally at least one pharmaceutically acceptable antioxidant and/or at least one pharmaceutically acceptable co-emulsifier as well as a first apparatus for mixing and/or dispersing, preferably stirring, the components in the first vessel,

b) a second vessel for providing a water phase 1 comprising the following components: water, optionally at least one pharmaceutically acceptable co-emulsifier and/or at least one substance for setting the pH value and/or at least one pharmaceutically acceptable preservative and/or at least one pharmaceutically acceptable isotonic agent, with the isotonic agent being present in a concentration of at most 18%t, preferably at most 15%, in relation to the total weight of the water phase 1, as well as a second apparatus for mixing and/or dispersing, preferably stirring, the components in the second vessel,

c) a tank for receiving the components from the first vessel via a first sterile filter and the components from the second vessel via a second sterile filter, with the tank having a third apparatus for mixing and/or dispersing, for producing a pre-emulsion by mixing the oil phase from the first vessel and the water phase 1 from the second vessel,

d) a homogenizer, preferably a high-pressure homogenizer, for producing a first emulsion by homogenizing the pre-emulsion,

e) a storage tank for providing a water phase 2 comprising the following components: water and optionally at least one pharmaceutically acceptable isotonic agent and/or at least one substance for setting the pH value and/or at least one pharmaceutically acceptable preservative as well as, if applicable, a third apparatus for mixing, preferably stirring, the components in the storage tank,

f) an apparatus for transferring the emulsion to the storage tank to produce the emulsion by mixing the first emulsion from the homogenizer and the water phase 2 from the storage tank and

g) an apparatus for sterilizing the emulsion,

h) an apparatus for filling the emulsion before or after being sterilized into a suitable container,

with at least one pharmaceutically acceptable emulsifier being added in the first vessel and/or in the second vessel and

with the water phase 1, provided in the second vessel, providing no more than 70%, preferably no more than 50%, particularly preferably no more than 30% and most preferably no more than 20% of the total amount of water contained in the emulsion.

According to the invention, the tank c) can be identical to the vessel a) or the vessel b) in the system for producing the oil-in-water emulsion.

### Use of the Oil-in-Water Emulsions Produced According to the Invention

The oil-in-water emulsions produced in accordance with the method according to the invention are administered preferably parenterally, particularly preferably intravenously, and are used as a medication or in the provision of parenteral nutrition.

### The Constituents

Where the concentration of the constituents is given in percent, the percentage information relates to mass proportions (mass/mass). Thus, “10% oil phase in relation to the total weight of the emulsion” means the same as “10 g oil phase per 100 g emulsion”.

### The Oils

The oil-in-water emulsions produced in accordance with the method according to the invention contain 1 to 40%, preferably 5 to 30%, most preferably 10 to 30%, for example 10%, 20% or 30% of an oil phase in relation to the total weight of the emulsion.

The oil phase can comprise a number of different oils, for example it comprises one or a plurality of oils selected from the group consisting of animal oils, such as fish oil, fish oil extract or krill oil, microbially produced oils, algae oils, fungal oils, synthetic or partially synthetic oils and vegetable oils, such as soybean oil, sunflower oil, coconut oil, olive oil, rapeseed oil, peanut oil, palm oil, sesame oil, safflower oil, almond oil, linseed oil or cotton seed oil.

Preferably, the oil phase comprises soybean oil, sunflower oil, coconut oil, medium-chain triglycerides (MCTs), olive oil, rapeseed oil, fish oil, fish oil extract, krill oil or mixtures thereof.

Particularly preferably, the oil phase comprises soybean oil, MCTs, olive oil, fish oil or mixtures thereof, for example mixtures of soybean oil and MCTs or mixtures of fish oil, soybean oil, olive oil and MCTs.

In a particularly preferred embodiment, the oil phase comprises 25 to 35%, preferably 30%, soybean oil, 25 to 35%, preferably 30%, MCTs, 20 to 30%, preferably 25%, olive oil and 10 to 20%, preferably 15%, fish oil in relation to the total weight of the oil phase.

“Fish oil” is understood in the context of the present invention as “purified fish oil” and “purified fish oil rich in n-3 fatty acids” according to the European Pharmacopoeia 6.0. It contains at least 9% docosahexaenoic acid (DHA) and at least 13% eicosapentaenoic acid (EPA) as triglycerides in relation to the total weight of the fish oil.

The term “fish oil extract” refers to mixtures with high EPA and DHA contents, which are, for example, obtained from fish oil by supercritical liquid extraction and subsequent, e.g. chromatographic, purification. Alternatively, the oil can be extracted as described in U.S. Pat. No. 6,750,048. Further extraction and/or purification methods are described in WO2001/076715 and WO2001/076385. Fish oil extract contains EPA and DHA in esterified form, for example in the form of their triglycerides or ethyl esters.

The term “medium-chain triglycerides” refers to the triglycerides of fatty acids with a chain length of 6 to 12 carbon atoms, for example caprylic acid, caproic acid, capric acid and lauric acid.

### The Water

Since the oil-in-water emulsions produced in accordance with the method according to the invention are preferably administered parenterally, the water used for providing the water phases 1 and 2 is preferably water for injection purposes (WFI).

### The Emulsifier

The method according to the invention comprises the addition of at least one emulsifier. The term “emulsifier” refers to amphiphilic substances, which stabilize the emulsion by reducing the interfacial tension between the oil and the water phase.

The emulsifier can be any pharmaceutically acceptable emulsifier suitable for producing oil-in-water emulsions. Suitable emulsifiers are lecithins, chemically modified lecithins (e.g. hydrated and/or ethoxylated lecithins), phospholipids, sphingolipids, sterols (e.g. cholesterol as well as derivatives and alkaline and alkaline earth salts of cholesterol, stigmasterol), bile acids and their salts (e.g. sodium cholate, sodium glycocholate, sodium taurocholate), block polymers and block-co-polymers (e.g. poloxamers, such as pluronic F-68, F-127 and poloxamines, such as tetronic 1304), polyglycerin ethers, polyglycerin esters, esters of sugars with fatty acids and/or fatty alcohols (e.g. sucrose monostearate, glycerin monooleate) and ethoxylated sorbitan fatty acid esters (e.g. Tween 20, 40, 60, 80).

They are used in concentrations of 0.1 to 5%, preferably 0.6 to 3% in relation to the total weight of the emulsion.

Preferably, the emulsifier is lecithin, which can be of animal (for example from krill or egg yolk) or plant (for example soybean lecithin) origin. The emulsifier most preferred according to the invention is egg lecithin.

Egg lecithin is used preferably in concentrations of 0.3 to 2.5%, preferably in concentrations of 0.6 to 1.5% in relation to the total weight of the emulsion.

### The Co-Emulsifier

The method according to the invention can comprise the addition of at least one co-emulsifier. The term “co-emulsifier” refers to amphiphilic substances, which stabilize the emulsion by reducing the interfacial tension between the oil and the water phase and, together with the emulsifier, accumulate at the phase boundary. Unlike the emulsifier, the co-emulsifier alone does not have to be suitable for forming self-associated structures, such as for example micelles. The co-emulsifier is normally used in smaller concentrations than the emulsifier.

Suitable co-emulsifiers are, for example, saturated and unsaturated fatty acids and their salts.

They are used in concentrations of 0.005 to 1% in relation to the total weight of the emulsion.

The co-emulsifier is preferably an unsaturated, preferably a long-chain monounsaturated fatty acid or an alkaline salt thereof, most preferably oleic acid or sodium oleate. The amount of co-emulsifier used is preferably between 0.01 and 1%, particularly preferably between 0.02 and 0.5% in relation to the total weight of the emulsion.

### The Co-Solvent

The method according to the invention can comprise the addition of at least one co-solvent. The term “co-solvent” refers to molecules, which can improve the stability of the emulsions produced in accordance with the method according to the invention. They reduce the dielectric constants of the water and make its environment more hydrophobic. Additionally, co-solvents increase the amount of molecularly dispersed emulsifier in the water phase. The availability of free emulsifier supports the solubilization of hydrophobic molecules.

Suitable co-solvents are, for example, ethanol, propylene glycol (1,2-propanediol), polyethylene glycols (PEG) with a molecular weight of 100 to 20,000 grams per mole and polypropylene glycols (PPG) with a molecular weight of 180 to 7000 grams per mole.

They are used in concentrations of 0.1 to 2.0%, preferably 0.70 to 1.40%, particularly preferably 0.80 to 1.30%, and most preferably 0.90 to 1.20%, in relation to the total weight of the emulsion.

They are preferably added in step e) of the method according to the invention.

The co-solvent is preferably a PEG, particularly preferably PEG 200 or PEG 400.

The amount of PEG used is preferably between 0.7 and 1.4%, particularly preferably between 0.9 and 1.2%, in relation to the total weight of the emulsion.

### The Isotonic Agent

The method according to the invention can comprise the addition of at least one pharmaceutically acceptable isotonic agent.

Suitable isotonic agents are salts (for example sodium chloride), polyols (for example mannitol or glycerin) and sugars (for example lactose or glucose).

They are used in concentrations of 0.1 to 10%, preferably 0.5 to 5%, particularly preferably of 0.7 to 3% in relation to the total weight of the emulsion.

The isotonic agent is preferably a polyol, particularly preferably glycerin.

The glycerin is preferably used in amounts of 1 to 5%, particularly preferably 1 to 3%, most preferably 2 to 2.5% in relation to the total weight of the emulsion.

The osmolality of the emulsions produced in accordance with the method according to the invention is preferably between 305 and 420 mOsmol/kg, measured with a vapor pressure osmometer, Model 5520 (Vapro™) according to USP 785.

### The Antioxidant

The method according to the invention can comprise the addition of at least one antioxidant. The antioxidant can be any pharmaceutically acceptable substance with antioxidative effect. Examples of suitable antioxidants are sodium metasulfite, sodium bisulfite, sodium sulfite, sodium thiosulfite, thioglycerol, thiosorbitol, thioglycolic acid, cysteine (preferably as cysteine hydrochloride), n-acetyl cysteine, citric acid, alpha-Tocopherol, beta-Tocopherol, gamma-Tocopherol, hydrophilic derivatives of vitamin E, lipophilic derivatives of vitamin E (e.g. vitamin E acetate), butylated hydroxyanisole (BHA), butylated hydroxytoluol (BHT), t-butylhydroquinone (TBHQ), monothioglycerin, propyl gallate, histidine, coenzymes Q10, tocotrienols, carotenoids, quinones, bioflavonoids, polyphenols, ascorbic acid (vitamin C) and ascorbic acid derivatives (e.g. ascorbyl palmitate, isoascorbic acid) and uric acid.

The antioxidant is used in concentrations of 0.001 to 0.5%, preferably 0.01 to 0.3%, in relation to the total weight of the emulsion.

The antioxidant is preferably selected from the group consisting of alpha-Tocopherol, beta-Tocopherol, gamma-Tocopherol and mixtures thereof.

The antioxidant is most preferably alpha-Tocopherol.

Alpha-Tocopherol is preferably used in concentrations of 0.01 to 0.3%, particularly preferably 0.05 to 0.2%, in relation to the total weight of the emulsion.

### The Substance for Setting the pH Value

The method according to the invention can comprise the addition of at least one substance for setting the pH value.

The substance for setting the pH value can be any pharmaceutically suitable acid or base.

Suitable acids are citric acid, lactic acid, phosphoric acid and hydrochloric acid (HCl).

The acid is preferably diluted hydrochloric acid, particularly preferably 0.1 M or 1 M hydrochloric acid.

Suitable bases are alkaline and alkaline earth bases.

The base is preferably sodium hydroxide and is used in the form of an aqueous solution (caustic soda).

The substance for setting the pH value is most preferably 0.1 M or 1 M caustic soda.

### The Preservative

The method according to the invention can comprise the addition of at least one pharmaceutically acceptable preservative.

Suitable preservatives are p-hydroxybenzoic acid as well as derivatives and salts of p-hydroxybenzoic acid, sorbic acid as well as derivatives and salts of sorbic acid, benzyl alcohol, chlorobutanol, thiomersal, chlorhexidine and its salts, phenyl mercuric salts, chlorocresol, ethylenediaminetetraacetic acid and its salts and phenoxyethanol.

They are used in concentrations of 0.001 to 2.0% in relation to the total weight of the emulsion.

The preservative is preferably ethylenediaminetetraacetic acid (EDTA) or a salt thereof and is used in concentrations of 0.05 to 0.8%, preferably 0.1 to 0.7%, in relation to the total weight of the emulsion.

### The Container

The emulsions produced in accordance with the method according to the invention are filled into a suitable container before or after being sterilized.

Suitable containers are bottles, syringes, ampules, vials, cans or bags. They can consist of any suitable material, for example of glass, metal, composite materials or plastic, and, if applicable, be coated, for example with plastic or silicone.

The suitable container is preferably a bottle of glass or plastic, a syringe of glass or plastic, a vial of glass or plastic or a plastic bag.

The emulsion obtained in step d) of the method according to the invention can also be filled into a suitable container before it is processed further.

Suitable containers comprise sterile intermediate bulk containers, for example of steel, stainless steel or plastic.

### Step a)

Providing the oil phase comprises mixing the different oils, if a mixture of oils is used, as well as optionally the addition of at least one emulsifier and/or at least one co-emulsifier and/or at least one antioxidant.

Providing the oil phase can also comprise the addition of at least one drug, preferably of a lipophilic drug, which is dissolved, suspended or dispersed, preferably dissolved, in the oil phase.

The lipophilic drug can, for example, be clevidipine, docetaxel, paclitaxel, dexamethasone, diazepam, cyclosporine, etomidate, flurbiprofen, bupivacaine, amphotericin B or propofol.

In preferred embodiments, propofol is added to the oil phase.

Providing the oil phase can also comprise the addition of at least one vitamin or one vitamin derivative, preferably of a lipophilic vitamin or a lipophilic vitamin derivative, which is dissolved, suspended or dispersed, preferably dissolved, in the oil phase. Lipophilic vitamins are the vitamins A, D, E and K. A lipophilic derivatives is, for example, ascorbyl palmitate.

The oil phase is preferably provided by stirring and heating.

The oil phase is preferably heated to 40 to 90° C., preferably to 50 to 80° C.

If the emulsifier is added to the oil phase, then it is heated preferably to temperatures of between 70 and 80° C. in order to facilitate/accelerate the dissolving/dispersing of the emulsifier and/or of the co-emulsifier.

If the emulsifier is added to the water phase 1, then the oil phase is heated preferably to temperatures of between 50 and 60° C. so that in step c) it has the same temperature as the water phase 1.

### Step b)

The water phase 1, provided in step b) provides no more than 70%, preferably no more than 50%, particularly preferably no more than 30% and most preferably no more than 20% of the total amount of water contained in the emulsion.

The water phase 1 preferably provides no less than 1%, 2% or 3% of the total amount of water contained in the emulsion.

Providing the water phase 1 can comprise mixing the water with at least one emulsifier and/or with at least one co-emulsifier and/or with at least one preservative.

It can also comprise setting the pH value, preferably to values between 6.0 and 10.0, in particular between 7.0 and 9.0, particularly preferably between 8.0 and 9.0.

Additionally, providing the water phase 1 can comprise the addition of at least one pharmaceutically acceptable isotonic agent. The concentration of the isotonic agent should not exceed 18%, preferably 15%, particularly preferably 14.3%, in relation to the total weight of the water phase 1.

Providing the water phase 1 can also comprise the addition of at least one drug, preferably of a water-soluble drug, which is dissolved, suspended or dispersed, preferably dissolved, in the water phase 1.

Providing the water phase 1 can also comprise the addition of at least one vitamin, preferably of at least one water-soluble vitamin, which is dissolved, suspended or dispersed, preferably dissolved, in the water phase 1. Water-soluble vitamins are the vitamins B1, B2, B6, B12, folic acid, biotin and vitamin C.

The water phase 1 is preferably provided by stirring and heating. The stirring tool can be an internal or external high shear mixer (e.g. a rotor-stator system from the companies, IKA or Ystral).

The water phase 1 is preferably heated to 40 to 90° C., particularly preferably to 50 to 80° C.

If the emulsifier is added to the oil phase, then the water phase 1 is heated preferably to temperatures of between 70 and 80° C. so that in step c) it has the same temperature as the oil phase.

If the emulsifier is added to the water phase 1, then the water phase 1 is heated preferably to temperatures of between 50 and 60° C. in order to facilitate/accelerate the dissolving/dispersing of the emulsifier and/or of the co-emulsifier.

### Step c)

The pre-emulsion is provided by mixing the oil phase, provided in step a), with the water phase 1, provided in step b). The mixing is preferably carried out by stirring. The stirring tool can be an internal or external high shear mixer (e.g. a rotor-stator system from the companies, IKA or Ystral).

The emulsion can be heated by inputting shearing energy during the emulsion formation. The temperature is held (for example via heat exchangers) preferably between 50 to 65° C.

### Step d)

According to the invention, the emulsion is produced in step d) by high-pressure homogenization, ultrasonic treatment or by means of a counter-jet disperser. The emulsion is preferably produced by high-pressure homogenization.

The homogenization is carried out preferably at temperatures of 40 to 70° C., particularly preferably of 40 to 60° C., most preferably of 50 to 60° C.

The high-pressure homogenization can take place by means of all conventional high-pressure homogenizers, for example using devices of the Ariete type from the company, GEA.

The high-pressure homogenization preferably takes place over a plurality of, preferably 4 to 6, cycles in a 2-stage high-pressure homogenizer, preferably at 350 to 600 bar in stage 1 and at 0 to 150 bar in stage 2.

### Step e)

Providing the water phase 2 can comprise mixing the water with at least one pharmaceutically acceptable isotonic agent and/or with at least one substance for setting the pH value and/or with at least one pharmaceutically acceptable preservative and/or with at least one co-solvent.

It can also comprise setting the pH value, preferably to values between 6.0 and 10.0, in particular between 7.0 and 9.0, particularly preferably between 8.0 and 9.0.

At least one drug or vitamin, preferably a water-soluble drug or a water-soluble vitamin, can also be added to the water phase 2. The drug and/or the vitamin is/are dissolved, suspended or dispersed, preferably dissolved, in the water phase 2. Water-soluble vitamins are the vitamins B1, B2, B6, B12, folic acid, biotin and vitamin C.

The water phase 1 is preferably provided by stirring. The stirring tool can be an internal or external high shear mixer (e.g. a rotor-stator system from the companies, IKA or Ystral).

The water phase 2 is temperature-regulated preferably to 5 to 25° C., particularly preferably 10 to 20° C., most preferably to 10 to 15° C.

### Step f)

The emulsion is produced in step f) by mixing the emulsion obtained in step d) with the water phase 2, provided in step e), preferably by adding the emulsion to the water phase 2, which is preferably stored in a suitable tank/vessel.

The emulsion is preferably produced by stirring with an internal propeller stirrer.

The emulsion is preferably gassed with nitrogen such that the oxygen content of the emulsion is preferably below 0.5 mg/l.

### Step g)

The emulsion can be sterilized using all suitable methods, for example by radiating, autoclaving or gassing.

Sterilizing is preferably carried out by autoclaving. Autoclaving is preferably carried out for 8 to 21 minutes at a pressure of 2 bar and a temperature of 116 to 123° C.

The emulsion is preferably filled into one of the above-mentioned suitable containers before being autoclaved.

### Embodiments

1. A method for producing an oil-in-water emulsion, comprising a water phase and 1 to 40%, preferably 5 to 30%, most preferably 10 to 30% of an oil phase, in relation to the total weight of the emulsion, wherein the method comprises the following steps:
a) providing an oil phase, comprising one or a plurality of oils and optionally at least one pharmaceutically acceptable antioxidant and/or at least one pharmaceutically acceptable co-emulsifier,
b) providing a water phase 1, comprising water, optionally at least one pharmaceutically acceptable co-emulsifier and/or at least one substance for setting the pH value and/or at least one pharmaceutically acceptable preservative and/or at least one pharmaceutically acceptable isotonic agent, wherein the isotonic agent is present in a concentration of at most 18%, preferably 15%, particularly preferably at most 14.3%, in relation to the total weight of the water phase 1,
c) producing a pre-emulsion by mixing the oil phase, provided in step a), with the water phase 1, provided in step b),
d) producing a first emulsion by homogenizing the pre-emulsion, provided in step c),
e) providing a water phase 2, comprising water, optionally at least one pharmaceutically acceptable isotonic agent and/or at least one substance for setting the pH value and/or at least one pharmaceutically acceptable preservative,
f) producing the emulsion by mixing the first emulsion, provided in step d), with the water phase 2, provided in step e) and
g) sterilizing the emulsion, obtained in step f),
   wherein the emulsion is filled into a suitable container before or after being sterilized,
   wherein at least one pharmaceutically acceptable emulsifier is added in step a) and/or in step b), and
   wherein the water phase 1, provided in step b), provides no more than 70%, preferably no more than 50%, particularly preferably no more than 30% and most preferably no more than 20% of the total amount of water contained in the emulsion.
2. The method according to embodiment 1, wherein the emulsion is provided for parenteral administration and wherein the water used for providing the water phases in the steps b) and e) is preferably water for injection purposes (WFI).
3. The method according to embodiment 1 or 2, wherein the pharmaceutically acceptable emulsifier is added in a concentration of 0.1 to 5% in relation to the total weight of the emulsion.
4. The method according to one of the preceding embodiments, wherein the pharmaceutically acceptable emulsifier is lecithin.
5. The method according to embodiment 4, wherein the lecithin is added in step a).
6. The method according to embodiment 4, wherein the lecithin is added in step b).
7. The method according to one of the preceding embodiments, wherein the pharmaceutically acceptable co-emulsifier is sodium oleate and is added in step b).
8. The method according to one of the embodiments 1 to 6, wherein the pharmaceutically acceptable co-emulsifier is oleic acid and is added in step a).
9. The method according to one of the preceding embodiments, wherein the oil-in-water emulsion comprises a pharmaceutically acceptable isotonic agent.
10. The method according to one of the preceding embodiments, wherein the pharmaceutically acceptable isotonic agent is a polyol, preferably glycerin.
11. The method according to one of the preceding embodiments, wherein the pharmaceutically acceptable isotonic agent is added in step b).
12. The method according to one of the preceding embodiments, wherein the pharmaceutically acceptable isotonic agent is added in step b) and in step e).
13. The method according to one of the preceding embodiments, wherein the isotonic agent is exclusively added in step e).
14. The method according to one of the preceding embodiments, wherein the oil phase comprises one or a plurality of oils selected from the group consisting of animal oils, microbially produced oils, algae oils, fungal oils, synthetic or partially synthetic oils and vegetable oils.
15. The method according to one of the preceding embodiments, wherein the emulsion comprises 10% or 20% of an oil phase in relation to the total weight of the emulsion.
16. The method according to one of the preceding embodiments, wherein the oil phase comprises at least one vegetable oil and/or at least one animal oil.
17. The method according to one of the preceding embodiments, wherein the oil phase comprises soybean oil, medium-chain triglycerides, olive oil, structured lipids, fish oil, fish oil extract, krill oil or mixtures thereof.
18. The method according to one of the preceding embodiments, wherein the oil phase comprises soybean oil, medium-chain triglycerides, olive oil, fish oil, fish oil extract or mixtures thereof.
19. The method according to one of the preceding embodiments, wherein the oil phase comprises soybean oil, medium-chain triglycerides, olive oil and fish oil.
20. The method according to one of the preceding embodiments, wherein the oil phase comprises 25 to 35%, preferably 30%, soybean oil, 25 to 35%, preferably 30%, medium-chain triglycerides, 20 to 30%, preferably 25%, olive oil and 10 to 20%, preferably 15%, fish oil in relation to the total weight of the oil phase.
21. The method according to one of the preceding embodiments, wherein the pH value is set in step b) and/or in step e) with NaOH to a value between 6.0 and 10.0, preferably between 7.0 and 9.0, particularly preferably between 8.0 and 9.0.
22. The method according to one of the preceding embodiments, wherein the average diameter of the oil droplets in the first emulsion after being homogenized in step d) and in the emulsion, obtained in step f), and after being sterilized in step g), is between 100 and 500 nm, preferably between 150 and 450 nm.
23. The method according to one of the preceding embodiments, wherein the PFAT₅ value of the first emulsion after being homogenized in step d) and the emulsion, obtained in step f), before and after being sterilized in step g), is below 0.05%, preferably below 0.04%, particularly preferably below 0.03% and most preferably below 0.02%.
24. The method according to one of the preceding embodiments, wherein the average PFAT₅ value of the first emulsion after being homogenized in step d) and the emulsion, obtained in step f), before and after being sterilized in step g), is below 0.035%, preferably below 0.030%, particularly preferably below 0.025%, more preferably below 0.020%, even more preferably below 0.015% and most preferably below 0.010%.
25. The method according to one of the preceding embodiments, wherein, in step a) and/or in step b) and/or in step e) and/or in step f), at least one drug and/or at least one vitamin is/are added, wherein the drug added in step a) and/or the vitamin added in step a) is preferably lipophilic and the drug added in step b) and/or e) and/or the vitamin added in step b) and/or e) is preferably water-soluble.
26. The method according to one of the preceding embodiments, wherein, in step a), a drug, preferably a lipophilic drug, particularly preferably a lipophilic drug selected from the group consisting of clevidipine, docetaxel, paclitaxel, dexamethasone, diazepam, cyclosporine, etomidate, flurbiprofen, bupivacaine, amphotericin B or propofol, most preferably propofol, is added to the oil phase.
27. The method according to one of the preceding embodiments, wherein the homogenization in step d) is carried out over 4 to 6 cycles in a two-stage high-pressure homogenizer and wherein homogenization is carried out in stage 1 at 350 to 600 bar and in stage 2 at 0 to 150 bar.
28. The method according to one of the preceding embodiments, wherein the oil phase, provided in step a), and the water phase 1, provided in step b), are temperature-regulated to 40 to 90° C., preferably to 50 to 80° C., before the pre-emulsion is produced in step c).
29. The method according to embodiment 28, wherein the oil phase, provided in step a), and the water phase 1, provided in step b), are temperature-regulated to 70 to 80° C., before the pre-emulsion is produced in step c).
30. The method according to embodiment 28, wherein the oil phase, provided in step a), and the water phase 1, provided in step b), are temperature-regulated to 55 to 65° C., before the pre-emulsion is produced in step c).
31. The method according to one of the preceding embodiments, wherein the water phase 2 in step e) is temperature-regulated to 5 to 25° C., preferably to 10 to 20° C., particularly preferably to 10 to 15° C.
32. The method according to one of the preceding embodiments, wherein the emulsion is filled into a suitable container before being sterilized and wherein the sterilization is preferably carried out by autoclaving.
33. The method according to one of the preceding embodiments, wherein the suitable container is a glass bottle, a syringe of plastic or glass or a plastic bag.
34. An oil-in-water emulsion, comprising a water phase and 1 to 40%, preferably 5 to 30%, most preferably 10 to 30%, of an oil phase in relation to the total weight of the emulsion, obtainable in accordance with the method according to one of the embodiments 1 to 32.
35. The oil-in-water emulsion according to embodiment 34 for use as a medication.
36. The oil-in-water emulsion according to one of the embodiments 34 or 35 for use in the provision of parenteral nutrition.
37. The oil-in-water emulsion, obtained in step d) of the method according to one of the embodiments 1 to 30.
38. A use of the oil-in-water emulsion according to embodiment 37 for further processing according to the steps e), f) and g) of the method according to one of the embodiments 1 to 33.
39. A system for producing an oil-in-water emulsion, comprising a water phase and 1 to 40%, preferably 5 to 30%, most preferably 10 to 30% of an oil phase in relation to the total weight of the emulsion, comprising
a) a first vessel for providing an oil phase comprising the following components: one or a plurality of oils, preferably selected from the group consisting of animal oils, microbially produced oils, algae oils, fungal oils, synthetic or partially synthetic oils and vegetable oils and optionally at least one pharmaceutically acceptable antioxidant and/or at least one pharmaceutically acceptable co-emulsifier as well as a first apparatus for mixing and/or dispersing, preferably stirring, the components in the first vessel,
b) a second vessel for providing a water phase 1 comprising the following components: water, optionally at least one pharmaceutically acceptable co-emulsifier and/or at least one substance for setting the pH value and/or at least one pharmaceutically acceptable preservative and/or at least one pharmaceutically acceptable isotonic agent, wherein the isotonic agent is present in a concentration of at most 18%, preferably at most 15%, in relation to the total weight of the water phase 1, as well as a second apparatus for mixing and/or dispersing, preferably stirring, the components in the second vessel,
c) a tank for receiving the components from the first vessel via a first sterile filter and the components from the second vessel via a second sterile filter, wherein the tank has a third apparatus for mixing and/or dispersing, for producing a pre-emulsion by mixing the oil phase from the first vessel and the water phase 1 from the second vessel,
d) a homogenizer, preferably a high-pressure homogenizer, for producing a first emulsion by homogenizing the pre-emulsion,
e) a storage tank for providing a water phase 2 comprising the following components: water, optionally at least one pharmaceutically acceptable isotonic agent and/or at least one substance for setting the pH value and/or at least one pharmaceutically acceptable preservative as well as, if applicable, a third apparatus for mixing, preferably stirring, the components in the storage tank,
f) an apparatus for transferring the emulsion to the storage tank to produce the emulsion by mixing the first emulsion from the homogenizer and the water phase 2 from the storage tank and
g) an apparatus for sterilizing the emulsion,
h) an apparatus for filling the emulsion before or after being sterilized into a suitable container, wherein at least one pharmaceutically acceptable emulsifier is added in the first vessel and/or in the second vessel and wherein the water phase 1, provided in the second vessel, provides no more than 70%, preferably no more than 50%, particularly preferably no more than 30% and most preferably no more than 20% of the total amount of water contained in the emulsion.

### EXAMPLES

### Example 1

It was determined in a series of tests up to which phase-volume ratio (amount of the internal phase, here thus the oil phase, in relation to the total of water phase 1 and oil phase), the water phase 1 can be reduced and what impact the concentration of the isotonic agent (here: glycerin) has.

The method carried out in test I corresponds to the known standard method.

TABLE 1

| Test number | I | II | III | IV | V | VI | VII |
|---|---|---|---|---|---|---|---|
| Emulsion formation | Yes | Yes | Yes | Yes | No | Yes | Yes |
| Water phase 1 | | | | | | | |
| WFI (g) | 75 | 35 | 20 | 15 | 10 | 10 | 10 |
| Glycerin (g) | 2.5 | 2.5 | 2.5 | 2.5 | 2.5 | 0 | 1.5 |
| Egg lecithin (g) | 1.2 | 1.2 | 1.2 | 1.2 | 1.2 | 1.2 | 1.2 |
| Sodium oleate (g) | 0.3 | 0.3 | 0.3 | 0.3 | 0.3 | 0.3 | 0.3 |
| 1M NaOH | q.s. | q.s. | q.s. | q.s. | q.s. | q.s. | q.s. |
| Oil phase | | | | | | | |
| Oil mixture (g) | 20 | 20 | 20 | 20 | 20 | 20 | 20 |
| alpha-Tocopherol (g) | 0.02 | 0.02 | 0.02 | 0.02 | 0.02 | 0.02 | 0.02 |
| Water phase 2 | | | | | | | |
| Water (g) | 0 | 40 | 55 | 60 | 70 | 70 | 70 |
| Glycerin (g) | 0 | 0 | 0 | 0 | 0 | 2.5 | 1 |
| Total amount (g) | 100 | 100 | 100 | 100 | 100 | 100 | 100 |
| Phase-volume | 0.21 | 0.35 | 0.47 | 0.53 | 0.62 | 0.67 | 0.63 |
| Ratio | | | | | | | |
| Glycerin concentration | 3.2 | 6.7 | 11.1 | 14.3 | 20 | 0 | 13 |
| Water phase 1 | | | | | | | |

The oils (a mixture of soybean oil, MCTs, olive oil and fish oil according to example 2 or 3) have been temperature-regulated to 55 to 60° C., the antioxidant (alpha-Tocopherol) was added and stirred for a further 15 minutes.

Parallel to this, the amounts of water for injection purposes (WFI) indicated in Table 1, the amounts of glycerin indicated in the Table and the amounts of caustic soda required for setting the pH value to 8.5 to 8.75 were each weighed in another vessel, temperature-regulated to 55 to 65° C. and the emulsifier (egg lecithin) and the co-emulsifier (sodium oleate) were successively added and dispersed in the WFI (water phase 1).

The heated oil phase was incorporated into the water phase 1 via a sterile filter and mixed for a further 30 minutes.

The remaining WFI and glycerin (water phase 2) were stored in a tank in the amounts indicated in Table 1 and temperature-regulated to 5 to 15° C.

The pre-emulsion was high-pressure homogenized with a MF-110F microfluidizer from the company, Microfluidics (4 cycles at 400/100 bar) and added to the storage tank with the WFI or the polyol/WFI mixture.

It has been surprisingly found that the water phase 1 can be reduced at least up to a phase-volume ratio of 0.67 (test IV). The emulsion formation failed with a glycerin concentration of 20% in the water phase 1 in the homogenization step.

The emulsion formation succeeded with glycerin concentrations of up to 14.3% in the water phase 1 in the emulsifying step (test IV). The polyol concentration in the emulsifying step should thus be below 18%, preferably below 15%, in relation to the total weight of the water phase 1.

### Example 2

Using the raw materials indicated in Table 2, 157 batches of an emulsion were produced according to the conventional production method (A) and 156 batches were produced in accordance with the method (B) according to the invention.

### Method A)

The soybean oil, the MCT oil, the olive oil and the fish oil were combined and temperature-regulated to 55 to 60° C.; then, the antioxidant (alpha-Tocopherol) was added and it was stirred for a further 15 minutes.

Parallel to this, 240 kg WFI and the amount of caustic soda required for setting a pH value of 8.5 to 8.75 were weighed in another vessel, temperature-regulated to 55 to 65° C. and the glycerin, the egg lecithin and the sodium oleate were added successively and dispersed in the WFI (water phase 1).

The heated oil phase was incorporated into the water phase 1 via a sterile filter and mixed for a further 30 minutes.

The raw emulsion was high-pressure homogenized with a Rannie 12.51H high-pressure homogenizer from the company, APV (4 cycles at 400/100 bar). Then, the pH value was re-set to a value between 8.5 to 8.75 and the water content was also set.

TABLE 2

| Raw material | Amount (kg) A | Amount (kg) B |
|---|---|---|
| Water for injection purposes (WFI) | 240.00 | 148.50 |
| Glycerin | 25.00 | 25.00 |
| Egg lecithin | 12.00 | 12.00 |
| Sodium oleate | 0.30 | 0.30 |
| NaOH IM | q.s. | q.s. |
| Soybean oil | 60.00 | 60.00 |
| MCT oil | 60.00 | 60.00 |
| Olive oil | 50.00 | 50.00 |
| Fish oil | 30.00 | 30.00 |
| alpha-Tocopherol | 0.02 | 0.02 |
| WFI | ad. 1000 | ad. 1000 |
| Nitrogen | g.s. | q.s. |

### Method B)

The soybean oil, the MCT oil, the olive oil and the fish oil were combined and temperature-regulated to 55 to 60° C.; then, the antioxidant was added and it was stirred for a further 15 minutes.

Parallel to this, 148.50 kg WFI and the amount of caustic soda required to set a pH value of 8.5 to 8.75 were weighed in another vessel, temperature-regulated to 55 to 65° C. and the glycerin, the egg lecithin and the sodium oleate were added successively and dispersed in the WFI (water phase 1).

The heated oil phase was incorporated into the water phase 1 via a sterile filter and mixed for a further 30 minutes.

The remaining WFI was stored in a tank and temperature-regulated to 5 to 15° C.

The pre-emulsion was high-pressure homogenized with a Rannie 12.51H high-pressure homogenizer from the company, APV (4 cycles at 400/100 bar) and added to the storage tank with the glycerin/WFI mixture. Then, the pH value and the water content were set.

The PFAT₅ value was determined for each batch.

The following average values and standard deviations resulted:

Average PFAT₅ (method A, n=157): 0.035±0.021

Average PFAT₅ (method B, n=156): 0.006±0.004

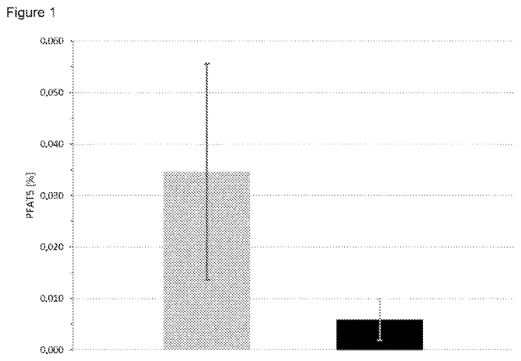

The method according to the invention has thus significantly reduced the average PFAT₅ value (see also FIG. 1).

None of the batches produced in accordance with the method according to the invention had to be destroyed because they had a PFAT₅ value of more than 0.05%. In particular, none of the batches produced in accordance with the method according to the invention had a PFAT₅ value of more than 0.017%.

The average droplet diameters (D₅₀) did not differ. However, the method B according to the invention leads to smaller deviations in the average droplet diameter.

The following droplet sizes have been measured (the average values and standard deviations are indicated):

D₅₀ (Method A): 351 nm±18 nm

D₅₀ (Method B): 353 nm±6 nm

### Example 3

TABLE 3

| Raw material | Amount (kg) |
|---|---|
| Water for injection purposes (WFI) | 115.00 |
| Glycerin | 25.00 |
| Egg lecithin | 12.00 |
| Sodium oleate | 0.30 |
| NaOH IM | q.s. |
| Soybean oil | 60.00 |
| MCT oil | 60.00 |
| Olive oil | 50.00 |
| Fish oil | 30.00 |
| alpha-Tocopherol | 0.02 |
| WFI | ad. 1000 |
| Nitrogen | q.s. |

The soybean oil, the MCT oil, the olive oil and the fish oil were combined and temperature-regulated to 55 to 60° C.; then, the antioxidant was added and it was stirred for a further 15 minutes.

Parallel to this, 115 kg WFI and the amount of caustic soda required for setting a pH value of 8.5 to 8.75 were weighed in another vessel, temperature-regulated to 55 to 65° C. and the egg lecithin and the sodium oleate were added successively and dispersed in the WFI (water phase 1).

The heated oil phase was incorporated into the water phase 1 via a sterile filter and mixed for a further 30 minutes.

The glycerin and the remaining WFI (water phase 2) were stored in a tank and temperature-regulated to 5 to 15° C.

The pre-emulsion was high-pressure homogenized with a Rannie 12.51H high-pressure homogenizer from the company, APV (4 cycles at 400/100 bar) and added to the storage tank with the glycerin/WFI mixture.

Then, the pH value and the water content were set.

Immediately after production, the average droplet diameter was 360 nm (D₅₀), PFAT₅ value was 0.006% and the content of non-esterified fatty acids (NEFA) was 2.0 mEq/L.

### Example 4

TABLE 4

| Raw material | Amount (g) |
|---|---|
| Water for injection purposes (WFI) | 99.00 |
| Glycerin | 25.00 |
| Egg lecithin | 12.00 |
| Oleic acid | 0.40 |
| NaOH 1M | q.s. |
| Soybean oil | 100.00 |
| MCT oil | 100.00 |
| Propofol | 10.00 |
| WFI | ad. 1000 |
| Nitrogen | q.s. |

Soybean oil, MCT oil, the oleic acid and propofol were combined, temperature-regulated to 55 to 65° C. and stirred for 15 minutes.

Parallel to this, 99 kg WFI and the amount of caustic soda required for setting a pH value of 8.0 to 8.75 were weighed in another vessel, temperature-regulated to 55 to 65° C. and the egg lecithin added and dispersed in the WFI (water phase 1).

The heated oil phase was incorporated into the water phase 1 via a sterile filter and mixed for a further 30 minutes.

The glycerin and the remaining WFI (water phase 2) were stored in a tank and temperature-regulated to 5 to 15° C.

The pre-emulsion was high-pressure homogenized with a MF-110F microfluidizer from the company, Microfluidics (4 cycles at 400/100 bar) and added to the storage tank with the glycerin/WFI mixture.

Then, the pH value (to 8.2) and the water content were set. The average droplet diameter was 236 nm (D₅₀).

### Example 5

TABLE 5

| Raw material | Amount (kg) |
|---|---|
| Water for injection purposes (WFI) | 76.00 |
| EDTA | 0.06 |
| Glycerin | 22.50 |
| Sodium oleate | 0.30 |
| Egg lecithin | 12.00 |
| NaOH 1M | q.s. |
| Soybean oil | 100.00 |
| Propofol | 10.00 |
| WFI | ad. 1000 |
| Nitrogen | q.s. |

The soybean oil was temperature-regulated to 73 to 77° C. The egg lecithin was added in portions and stirred for a further 15 minutes until all the egg lecithin was dissolved.

Parallel to this, 76 kg WFI and the amount of caustic soda required for setting a pH value of 8.5 to 9.5 were weighed in another vessel, and temperature-regulated to 73 to 77° C. Then, the EDTA as well as the sodium oleate were added and dissolved (water phase 1).

The heated oil phase was incorporated into the water phase 1 via a sterile filter and mixed for a further 30 minutes.

The glycerin and the remaining WFI (water phase 2) were stored in a tank and temperature-regulated to 5 to 15° C.

The pre-emulsion was high-pressure homogenized with a MF-110F microfluidizer from the company, Microfluidics (6 cycles at 490/0 bar) and added to the storage tank with the glycerin/WFI mixture.

Then, the water content and the pH value were set.

The average droplet diameter was 267 nm (D₅₀).

### Example 6

TABLE 6

| Raw material | Amount (kg) |
|---|---|
| Water for injection purposes (WFI) | 140.00 |
| Glycerin | 22.00 |
| Sodium oleate | 0.30 |
| Egg lecithin | 12.00 |
| NaOH 1M | q.s. |
| Soybean oil | 200.00 |
| WFI | ad. 1000 |
| Nitrogen | q.s. |

The soybean oil was temperature-regulated to 73 to 77° C. The egg lecithin was added in portions and stirred for a further 15 minutes until all the egg lecithin was dissolved.

Parallel to this, 140 kg WFI, 0.30 kg sodium oleate and the amount of caustic soda required for setting a pH value of 8.5 to 9.5 were weighed in another vessel and temperature-regulated to 73 to 77° C. (water phase 1).

The heated oil phase was incorporated into the water phase 1 via a sterile filter and mixed for a further 30 minutes.

The glycerin and the remaining WFI (water phase 2) were stored in a tank and temperature-regulated to 5 to 15° C.

The pre-emulsion was high-pressure homogenized with a MF-110F microfluidizer from the company, Microfluidics (6 cycles at 560/120 bar) and added to the storage tank with the glycerin/WFI mixture.

Then, the water content and the pH value were set.

The average droplet diameter was 393 nm (D₅₀).

### DESCRIPTION OF THE IMAGE

FIG. 1 shows the average values (with standard deviations) of the PFAT₅ values (determined according to USP 729, method 2) of the emulsion batches obtained according to example 2. In this case, the gray bar represents the average value of the PFAT₅ values of the batches produced according to method A, while the black bar denotes the average value of the PFAT₅ values of the batches produced in accordance with the method B according to the invention.

1. A method for producing an oil-in-water emulsion, comprising a water phase and 1 to 40% of an oil phase, in relation to the total weight of the emulsion, wherein the method comprises the following steps:
a) providing an oil phase, comprising one or a plurality of oils selected from the group consisting of animal oils, microbially produced oils, algae oils, fungal oils, synthetic or partially synthetic oils and vegetable oils; and,
optionally a pharmaceutically acceptable antioxidant and/or at least one pharmaceutically acceptable co-emulsifier;
b) providing a water phase 1, comprising water and optionally at least one pharmaceutically acceptable co-emulsifier and/or at least one substance for setting the pH value and/or at least one pharmaceutically acceptable preservative and/or at least one pharmaceutically acceptable isotonic agent, wherein the isotonic agent is present in a concentration of at most 18%, in relation to the total weight of the water phase;
c) producing a pre-emulsion by mixing the oil phase, provided in step a), with the water phase 1, provided in step b);
d) producing a first emulsion by homogenizing the pre-emulsion, provided in step c);
e) providing a water phase 2, comprising water and optionally at least one pharmaceutically acceptable isotonic agent and/or at least one substance for setting the pH value and/or at least one pharmaceutically acceptable preservative;
f) producing the emulsion by mixing the first emulsion, provided in step d), with the water phase 2, provided in step e); and
g) sterilizing the emulsion, obtained in step f);
wherein the emulsion is filled into a suitable container before or after being sterilized;
wherein at least one pharmaceutically acceptable emulsifier is added in step a) and/or in step b); and
wherein the water phase 1, provided in step b), provides no more than 30% of the total amount of water contained in the emulsion.

2. The method according to claim 1, wherein the emulsion is provided for parenteral administration and wherein the water used for providing the water phases 1 and 2 in the steps b) and e) is water for injection purposes (WFI).

3. The method according to claim 1, wherein the pharmaceutically acceptable emulsifier is added in a concentration of 0.1 to 5% in relation to the total weight of the emulsion.

4. The method according to claim 1, wherein the pharmaceutically acceptable emulsifier is lecithin.

5. The method according to claim 1, wherein the pharmaceutically acceptable co-emulsifier is sodium oleate and is added in step b).

6. The method according to claim 1, wherein the pharmaceutically acceptable co-emulsifier is oleic acid and is added in step a).

7. The method according to claim 1, wherein the oil-in-water emulsion comprises a pharmaceutically acceptable isotonic agent.

8. The method according to claim 1, wherein the pharmaceutically acceptable isotonic agent is a polyol.

9. The method according to claim 1, wherein the pharmaceutically acceptable isotonic agent is added in step b).

10. The method according to claim 1, wherein the pharmaceutically acceptable isotonic agent is present in step b) in a concentration of at most 15% in relation to the total weight of the water phase 1.

11. The method according to claim 1, wherein the isotonic agent is exclusively added in step e).

12. (canceled)

13. The method according to claim 1, wherein the oil phase comprises a vegetable oil and/or an animal oil.

14. The method according to claim 1, wherein the oil phase comprises soybean oil, medium-chain triglycerides, olive oil, structured lipids, fish oil, fish oil extract, krill oil or mixtures thereof

15. (canceled)

16. The method according to claim 1, wherein the oil phase comprises soybean oil, medium-chain triglycerides, olive oil and fish oil.

17. The method according to claim 1, wherein the oil phase comprises 25 to 35% soybean oil, 25 to 35% MCTs, 20 to 30% olive oil and 10 to 20% fish oil in relation to the total weight of the oil phase.

18. (canceled)

19. (canceled)

20. The method according to claim 1, wherein the PFAT₅ value of the emulsion, obtained in step d), and the emulsion, obtained in step f), before and after being sterilized in step g) is below 0.05%.

21. The method according to claim 1, wherein the average PFAT₅ value of the emulsion, obtained in step d), and in the emulsion, obtained in step f), before and after being sterilized in step g), is below 0.035%.

22. The method according to claim 1, wherein one or a plurality of drugs and/or one or a plurality of vitamins are added in step a) and/or in step b) and/or in step e) and/or in step f).

23-27. (canceled)

28. An oil-in-water emulsion, comprising a water phase and 1 to 40% of an oil phase in relation to the total weight of the emulsion, obtained in accordance with the method according to claim 1.

29. (canceled)

30. An oil in water emulsion said emulsion prepared according to a method comprising a water phase and 1 to 40%, in relation to the total weight of the emulsion, wherein the method comprises the following steps:
a) providing an oil phase, comprising one or a plurality of oils selected from the group consisting of animal oils, microbially produced oils, algae oils, fungal oils, synthetic or partially synthetic oils and vegetable oils; and,
optionally a pharmaceutically acceptable antioxidant and/or at least one pharmaceutically acceptable co-emulsifier;
b) providing a water phase 1, comprising water and optionally at least one pharmaceutically acceptable co-emulsifier and/or at least one substance for setting the pH value and/or at least one pharmaceutically acceptable preservative and/or at least one pharmaceutically acceptable isotonic agent, wherein the isotonic agent is present in a concentration of at most 18%, in relation to the total weight of the water phase;
c) producing a pre-emulsion by mixing the oil phase, provided in step a), with the water phase 1, provided in step b);
d) producing an emulsion by homogenizing the pre-emulsion, provided in step c).
